(19) United States
(12) Patent Application Publication
(10) Pub. No.: US 2007/0180904 A1
(43) Pub. Date: Aug. 9, 2007

(54) Fluid level sensor

(75) Inventor: Shawn X. Gao, Irvine, CA (US)

Correspondence Address: ALCON, IP LEGAL, TB4-8, 6201 SOUTH FREEWAY, FORT WORTH, TX, 76134, US

(73) Assignee: Alcon, Inc.

(21) Appl. No.: 11/347,868

(22) Filed: Feb. 6, 2006

(51) Int. Cl. G01F 23/24; A61B 19/00

(52) U.S. Cl. 73/304 R; 604/404

(57) ABSTRACT

A fluid level sensor for use in a chamber contained in a surgical cassette. The sensor has a pair of parallel conductor that project vertically into the chamber forming an open circuit. When surgical fluid contained within the chamber touches the conductor, the circuit is completed and AC voltage or current is passed through the conductor circuit. The conductivity of the circuit can be measured and related to the depth of fluid covering the conductors using laboratory-generated data or in situ calibration of the cassette.

### BACKGROUND OF THE INVENTION

The present invention relates to an apparatus for sensing the level of fluid within a surgical cassette that is one component of an ophthalmic surgical instrument.

Conventional ophthalmic surgical instrument systems use vacuum to aspirate the surgical site and positive pressure to irrigate the site. Typically, a cassette is serially connected between the means used to generate pressure and the surgical instrument. The use of cassettes with surgical instruments to help manage irrigation and aspiration flows at a surgical site is well known. U.S. Pat. Nos. 4,493,695 and 4,627,833 (Cook), U.S. Pat. No. 4,395,258 (Wang, et al.), U.S. Pat. No. 4,713,051 (Steppe, et al.), U.S. Pat. No. 4,798,850 (DeMeo, et al.), U.S. Pat. Nos. 4,758,238, 4,790,816 (Sundblom, et al.), and U.S. Pat. Nos. 5,267,956, 5,364,342 (Beuchat) and U.S. Pat. No. 5,747,824 (Jung, et al.) all disclose ophthalmic surgical cassettes with or without tubes, and they are incorporated in their entirety by this reference. Aspiration fluid flow rate, pump speed, vacuum level, irrigation fluid pressure, and irrigation fluid flow rate are some of the parameters that require precise control during ophthalmic surgery.

For aspiration instruments, the air pressure in the cassette is below atmospheric pressure, and fluid within the cassette has been removed from the surgical site. For irrigation instruments, the air pressure in the cassette is higher than atmospheric pressure, and the fluid will be transported to the surgical site. In both types of instruments, the cassette acts as a reservoir for the fluid that buffers variations caused by the pressure generation means.

For the cassette to act as an effective reservoir, the level of fluid (and thus the empty volume) within the cassette must be controlled so that the cassette is neither completely filled nor emptied. If fluid fills the cassette in an aspiration system, fluid may be drawn into the means for generating vacuum (typically a venturi), which would unacceptably interfere with the vacuum level at the surgical instrument. An empty cassette in an aspiration system will result in air being pumped into the drain bag, which would waste valuable reservoir space within the bag. Moreover, constant volume within the cassette in an aspiration system enables more precise control of the level of vacuum within the surgical instrument. Control of the fluid level within cassettes of irrigation systems is similarly desirable.

Additionally, the size of the reservoir within the cassette affect the response time of the cassette. A larger reservoir provides more storage capacity but slows the response time of the system. A smaller reservoir increases the response time of the system, but may not have adequate storage capacity. This dilemma has been addressed by cassettes have two internal reservoirs. Such a cassette is illustrated in U.S. Pat. No. 4,758,238 (Sundblom, et al.) (the “Sundblom Cassette”). The smaller reservoir is in direct fluid communication with the surgical handpiece while a larger reservoir is positioned between the smaller reservoir and the source of vacuum. This allows for a faster response time and larger storage capacity. The smaller reservoir, however, must be periodically emptied into the larger reservoir prior to the smaller reservoir filling up. This requires that the smaller reservoir contain a fluid level sensor that notifies the control console to empty the smaller reservoir at the appropriate time. The Sundblom Cassette uses two electrical probes 76 (see FIG. 8) that form an open electrical alarm circuit. When the surgical fluid (which is electrically conductive) fills small reservoir 30, both probes 76 are submersed in the fluid, thereby closing the circuit and triggering the alarm that reservoir 30 is full. The fluid level sensor used in the Sundblom cassette has the limitation of being a simple “On/Off” switch. The sensor has no other function other than to trigger a “reservoir full” alarm and provides no other information to the user about the amount of fluid in the small reservoir.

Other level sensors, such at the one disclosed in U.S. Pat. No. 5,747,824 (Jung, et al.) use an optical device for continuous fluid level sensing by reading the location of the air/fluid interface. These optical devices require relatively expensive phototransmitters and receivers and are subject to inaccuracies due to foaming of the fluid within the reservoir.

Accordingly, a need continues to exist for a simple, reliable and accurate fluid level sensor.

### BRIEF DESCRIPTION OF THE INVENTION

The present invention improves upon the prior art by providing a fluid level sensor for use in a chamber contained in a surgical cassette. The sensor has a pair of parallel conductors that project vertically into the chamber forming an open circuit. When surgical fluid contained within the chamber touches the conductors, the circuit is completed and AC voltage or current is passed through the conductor circuit. The conductivity of the circuit can be measured and related to the depth of fluid covering the conductors using laboratory-generated data or in situ calibration of the cassette.

Accordingly, one objective of the present invention is to provide a fluid level sensor.

Another objective of the present invention is to provide a simple, reliable fluid level sensor.

Yet another objective of the present invention is to provide a sensor that continuously measures fluid level.

Yet another objective of the present invention is to provide a non-optical fluid level sensor.

Still another objective of the present invention is to provide a fluid level sensor using a pair of parallel conductors.

These and other advantages and objectives of the present invention will become apparent from the detailed description, drawings and claims that follow.

### BRIEF DESCRIPTION OF THE DRAWINGS

FIG. 1 is a schematic representation of a surgical cassette and console employing the fluid level sensor of the present invention.

FIG. 2 is a schematic representation of the fluid level sensor of the present invention with the fluid chamber empty.

FIG. 3 is a schematic representation of the fluid level sensor of the present invention with the fluid chamber having a small amount of surgical fluid contained therein.

FIG. 4 is a schematic representation of the fluid level sensor of the present invention with the fluid chamber chamber having a larger amount of surgical fluid, relative to FIG. 3, contained therein.

### DETAILED DESCRIPTION OF THE PREFERRED EMBODIMENTS

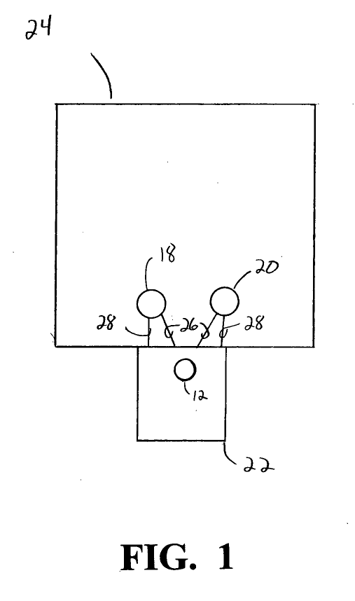

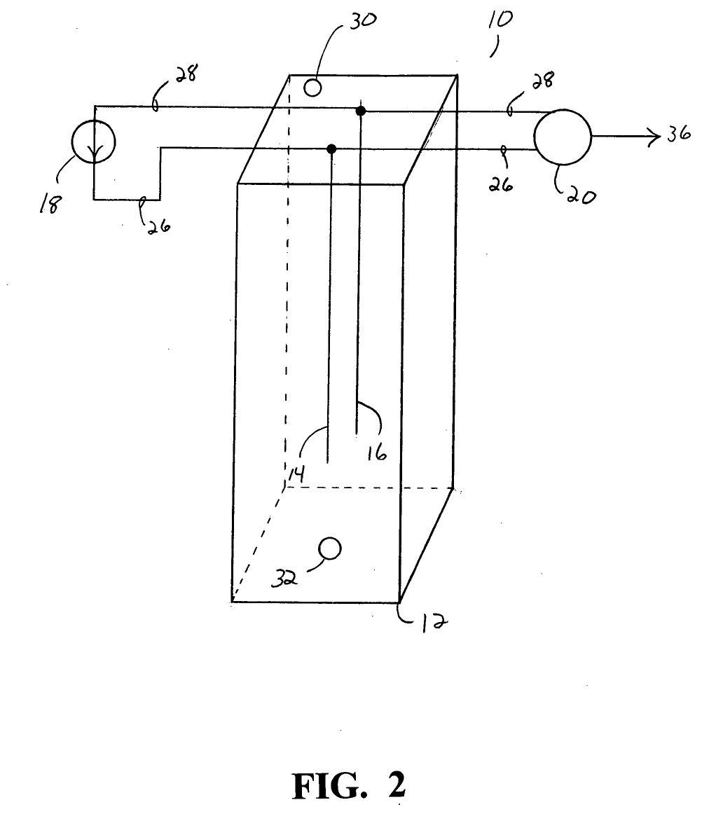

As best seen in FIGS. 1 and 2, fluid level sensor 10 of the present invention generally include fluid chamber 12, electrical conductor or electrodes 14 and 16, AC current source 18, and signal processor 20. Fluid chamber 12 generally is formed as part of cassette 22 while AC current source 18 and signal processor 20 generally are contained within surgical console 24 as part of the operating hardware and software of console 24. Conductors 14 and 16 are connected to AC current source 18 and signal processor 20 by appropriate wires or other conductors 26 and 28, respectively. AC current source 18 uses AC current instead of DC current to avoid electrolysis that may be caused by DC current. Signal processor 20 may be any suitable analog or digital signal processor as are well-known and used in surgical instrumentation.

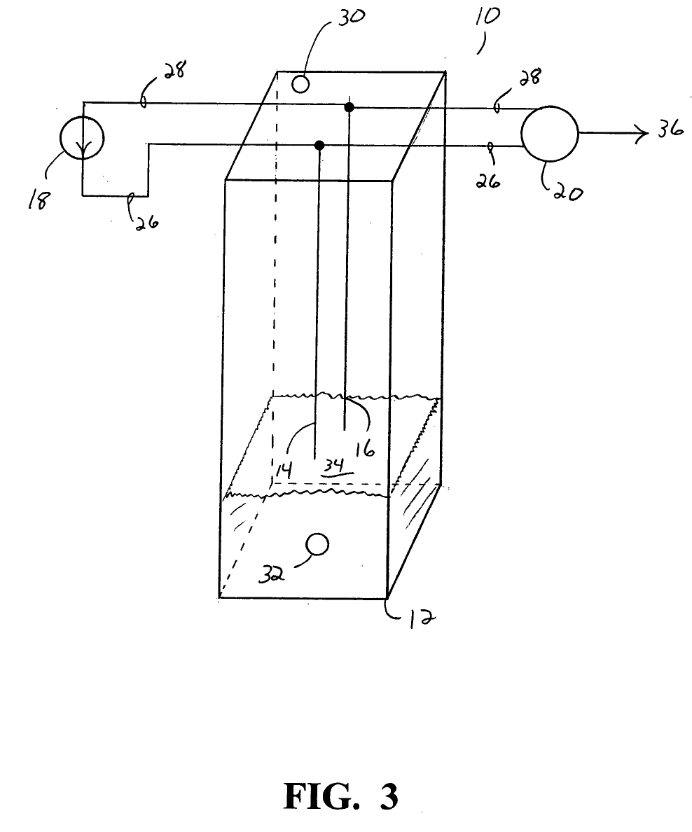

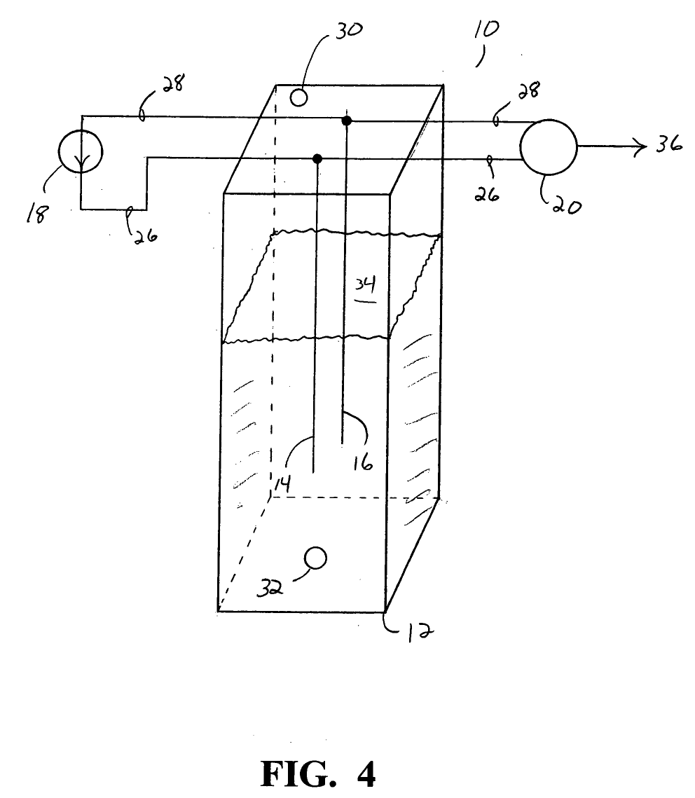

As best seen in FIGS. 3 and 4, in use, chamber 12 is filled with surgical fluid 34 (normally an electrically conductive saline solution) through inlet 30. As the level of fluid 34 within chamber 12 rises, it will contact conductors 14 and 16, as shown in FIG. 3. The continued introduction of fluid 34 into chamber 12 causes conductors 14 and 16 to be submerged (to a varying degree) within fluid 34, as shown in FIG. 4. The electrically conductive nature of fluid 34 closes the open circuit between conductors 14 and 16 and allows AC current or voltage for AC current source 18 to pass between conductors 14 and 16 and thereby provide an input signal to signal processor 20. The amount of voltage that is generated by passing current between conductors 14 and 16 will vary proportionally upon the amount that conductors 14 and 16 are submerged within fluid 34. Therefore, the signal sent to signal processor 20 will vary depending upon the level of fluid 34 within chamber 12. Signal processor 20 processes this signal and provides control output 36 that is used as control system input by surgical console 24. If necessary, surgical console 24 may drain chamber 12 through outlet 32.

This description is given for purposes of illustration and explanation. It will be apparent to those skilled in the relevant art that modifications may be made to the invention as herein described without departing from its scope or spirit.

I claim:

1. A fluid level sensor, comprising:
a) a fluid chamber having an inlet and an outlet
b) a pair of conductors projecting into the fluid chamber;
c) a source of AC current or voltage electrically connected to the conductors; and
d) a signal processor electrically connected to the conductors.

2. The fluid level sensor of claim 1 wherein the source of AC current or voltage provides an input signal to the signal processor when a surgical fluid is introduced into the chamber through the inlet.

3. The fluid level sensor of claim 1 wherein the signal processor provides a control signal to a surgical console.

4. The fluid level sensor of claim 2 wherein the input signal varies as a function of a level of surgical fluid in the chamber.

5. The fluid level sensor of claim 3 wherein the control signal varies as a function of a level of surgical fluid in the chamber.

6. A method of generating a control signal indicative of a level of fluid in a chamber, the method comprising the steps of:
a) providing a fluid chamber having an inlet, an outlet and a pair of conductor projecting into the chamber;
b) connecting a signal processor to the conductors;
c) introducing a surgical fluid into the chamber through the inlet in an amount sufficient to submerge the conductors in the fluid;
d) passing AC current or voltage through the conductors and the surgical fluid to generate a signal indicative of a relative submersion of the conductors in the surgical fluid; and
e) processing the signal by the signal processor to generate a control signal indicative of a level of the surgical fluid in the chamber.
